(19) United States
(12) Patent Application Publication
(10) Pub. No.: US 2003/0103913 A1
(43) Pub. Date: Jun. 5, 2003

(54) Metal activated tooth whitening compositions

(76) Inventor: Salim A. Nathoo, Piscataway, NJ (US)

Correspondence Address: ABRAHAM WILSON, 508 ELLIS PARKWAY, PISCATAWAY, NJ, 08854, US

(21) Appl. No.: 09/850,496

(22) Filed: May 7, 2001

(51) Int. Cl. A61K 7/20

(52) U.S. Cl. 424/53

(57) ABSTRACT

A two component aqueous whitening dentifrice composition is disclosed which comprises a first component containing a peroxygen compound such as hydrogen peroxide and a second dentifrice component containing an essentially insoluble transition metal oxide completed with metal chelating agents to form a peroxide activation complex, which activates the peroxygen compound and accelerates the release of active bleaching species for rapid whitening action, the first and second components being maintained separate from each other until dispensed for application to teeth.

### BACKGROUND OF THE INVENTION

1. Field of the Invention

This invention relates generally to an oral composition which when applied onto the surface of teeth acts to rapidly whiten teeth when compared to existing products.

2. The Prior Art

A tooth is comprised of an inner dentin layer and outer hard enamel that is coated with a protective layer called the acquired pellicle. The pellicle or the enamel can become stained or discolored. The enamel layer of a tooth is composed of hydroxyapatite mineral crystals that create a somewhat porous surface. It is believed that this porous nature of the enamel layer is what allows staining agents and discolor substances to permeate the enamel and discolor the tooth.

Many substances that a person confronts or comes in contact with On a daily basis can “stain” or reduce the “whiteness” of one's teeth. In particular, the foods, tobacco products and fluids such as tea and coffee that one consumes tend to stain one's teeth. These products or substances tend to accumulate on the enamel layer of the tooth and form a pellicle film over the teeth. These staining and discoloring substances can then permeate the enamel and causing noticeable discoloration of one's teeth.

There are available in the marketplace oral compositions for home use which contain 1-3% by weight concentrations of a peroxygen compound such as hydrogen peroxide and when applied on the teeth affect whitening of stains. However, these compositions are considered to have a slow bleaching effect.

Illustrative of oral compositions containing peroxygen compounds for whitening teeth include U.S. Pat. Nos. 5,597,554, 5,302,374, 5,279,816, 4,988,450; 4,980,152, 4,839,156, 4,405,599, 3,988,433 and 3,657,417.

U.S. Pat. No. 5,279,816 discloses an oral composition for whitening teeth containing peracetic acid dissolved or suspended in a vehicle. U.S. Pat. No. 5,302,374 discloses generating peracetic acid within a dentifrice vehicle by combining water, acetylsalicylic acid and a water soluble alkali metal percarbonate U.S. Pat. Nos. 4,988,450 and 3,657,417 disclose formulating oxygen liberating compositions for the whitening of teeth utilizing anhydrous pastes or gels.

U.S. Pat. No. 4,980,152 discloses an aqueous oral gel composition comprising about 0.5 to about 10% by weight urea peroxide and 0.01 to 2% by weight of a fluoride providing compound, U.S. Pat. No. 4,839,156 discloses water containing a hydrogen peroxide-Pluronic thickened oral gel composition.

U.S. Pat. No. 4,405,599 discloses toothpaste containg a combination of calcium peroxide and sodium perborate oxidizing agents, dicalcium phosphate, calcium carbonate and magnesium carbonate cleaning agents, sorbitol hurnectant, cornstarch and cellulose gum thickening agents, and an anionic detergent.

U.S. Pat. No. 3,988,433 discloses oral compositions containing peroxyacids and alkyl diperoxy acids having alkaline groups containing 5-11 carbon atoms which remove stain from teeth. The use of titanium dioxide as a pigmenting agent in oral care compositions is well known, illustrative examples include U.S. Pat. Nos. 6,106,812, 6,110,446, 5,112,365, 5,401,495, 5,041,280, 4,603,045, 5,902,568 and 5,648,064.

U.S. Pat. Nos. 5,302,374 and 5,597,554 disclose a conventional toothpaste containing titanium dioxide as one of the ingredients which is added to a hydrogen peroxide dentifrice to accelerate the breakdown of peroxide and produce oxygen. This combination of ingredients it thought to be useful in oral care compositions because oxygen is toxic to anaerobic organisms responsible for periodontal disease. The bubbling action of the oxygen is also thought to cleanse the tooth surfaces through a mechanical action. In contrast, U.S. Pat. Nos. 4,687,663, 4,849,213 and 4,983,379 discloses the use of titanium dioxide as a polishing agent/stabilizer/cleansing agent contained in the bicarbonate portion of the peroxide/bicarbonate dentifrice.

The reasons for the apparent contradictions between U.S. Pat. Nos. 5,302,374, 5,597,554 and U.S. Pat. Nos. 4,687,663, 4,849,213, 4,983,379 are not clear. However, to those skilled in bleaching technology it is known that the rapid degradation of peroxide results in reduced bleaching effectiveness because oxygen, in itself, is not a good bleaching agent. The agents responsible for bleaching are the free radicals such as the perhydroxy anion and the superoxide radical which are the products of peroxide activation.

U.S. Pat. Nos. 3,156,654 and 4,728,455 teach that heavy metals have an adverse effect upon bleaching performance of fabrics. To overcome the problems associated with the loss of bleaching performance U.S. Pat. No. 5,684,064 discloses the use of soluble manganese coordination complexes for activating peroxide in oral care compositions. The drawback with using manganese coordination complexes has been described in U.S. Pat. No. 6,002,490 which teach that manganese complex are not very effective at 20 to 40 deg. C. i.e., body temperature. Other metal activated peroxide compositions e.g., WO 101943A1 and WO 9964554A1 require external activation such as activation by radiant energy. The compositions described herein do not require external energy.

It is well known that compliance in a therapeutic regimen has been shown to be directly related to the length of the therapy and the frequency of dosage. Hence, it is the object of this invention to provide a composition which can rapidly whiten teeth by producing active bleaching species without undesirable peroxide decomposition into oxygen and water. It is also the object of this invention to provide with compositions which can be used in the home by the consumer or can be used in the dental office.

### SUMMARY OF THE INVENTION

The present invention is based upon the discovery that when a peroxide containing component and titanium dioxide component which are normally incompatible with each other and combined for the first time result in rapid whitening of teeth. It was unexpectedly found that when titanium dioxide or the substantially insoluble transition metal oxide is allowed to form complexes with the chelating agents and then the peroxide containing component is allowed to mix with the titanium dioxide-chelating agent complex, the ingredients contained in these components do not appreciable immediately react to decompose the peroxide into the less efficacious oxygen and water. Without being bound to a particular theory it is thought that the chelating agent reacts with titanium dioxide forming a peroxide activation complex. The resulting metal chelate or the peroxide activation complex participates a reaction whereby hydrogen peroxide is dissociated into active bleaching species such as hydroxyl radicals, perhydroxy anions or superoxide radicals rather then the expected oxygen and water. Hence, it is critical to the present invention that metal chelating agents are present in the titanium dioxide containing component and also the transition metal is preincubated with the metal chelating agents such that peroxide activation complexes are formed.

For the purposes of definition substantially insoluble is defined as a solubility of less then 1 g in 10 ml of water at room temperature.

The peroxide activation complex is defined here as a metal complex consisting of a metal chelating agent-insoluble transition metal compound complex which is capable of activating the peroxygen compound in a manner that produces active bleaching species.

In accordance with the present invention there is provided a peroxygen oral composition for accelerated whitening of teeth wherein there is provided a two component composition of separate unmixed phases comprising of:

(a) a first component containing a water soluble peroxygen compound contained in an orally acceptable vehicle.

(b) a second component containing an insoluble titanium compound combined with a metal chelating agent in an orally acceptable vehicle in an amount effective to activate the peroxygen compound and accelerate the release of active bleaching species.

The two phases are combined shortly before application to the teeth wherein the peroxide activation complex interacts with the peroxygen constituent to accelerate the rapid release of the active bleaching species from the peroxygen compound, such rapid release being effective for whitening teeth. The present invention offers the advantages that the premature breakdown of the peroxygen compound is avoided and the active bleaching species are generated quickly and in large quantities thereby facilitating convenient and effective home use by the consumer as well as use by the professional, such as dentist performed tooth whitening. DETAILED DESCRIPTION OF THE INVENTION Peroxygen compounds useful in the oral compositions of the invention include hydrogen peroxide, urea peroxide, metal peroxides such as calcium peroxide, sodium peroxide, strontium peroxide, magnesium peroxide, and the salts of perbotate, persilicate perphosphate and percarbonate such as sodium perborate, potassium persilicate and sodium percarbonate. The most suitable peroxygen compound for this invention is hydrogen peroxide.

Substantially insoluble transition metal compounds for use as activator compounds in the practice of the present invention include compounds of transition metals from atomic #19 to atomic #32. The preferred compounds are transition metal oxides from atomic #19 to atomic #32. The most preferred transition metal activator is titanium dioxide.

The amount of peroxygen compound incorporated in the first component of the two component oral composition of the present invention will vary dependent upon its intended use. For use by trained professionals in office treatments or dentist-monitored treatments, the concentration of peroxygen compound incorporated in the oral composition can vary from about 3 to about 30% by weight. For home use applications such as tooth brushing, the typical consumer cannot use such high concentrations of peroxygen compounds safely and therefore the useful range of peroxygen compound when the oral composition is a paste, gel or rinse is between 0.1 to 3.0% by weight. The preferred range is between about 0.5 to about 2.0% by weight.

The amount of titanium dioxide activator compound present in the second component of the two phase whitening oral composition of the present invention will vary depending upon the amount of peroxygen compound incorporated in the first component. When the whitening oral composition is to be used by trained professionals and the first component contains relatively high concentrations of a peroxygen compound, e.g. 3 to 35% by weight, the amount of titanium dioxide activator compound incorporated in the second component will range between 0.1 to 6% by weight and preferably between 0.25 to 4% by weight.

For home use oral compositions in which the concentration range of peroxygen compound in the first oral composition component is between about 0.1 to about 3.0% by weight, lower concentrations, e.g., between about 0.001 to about 2% by weight of the titanium activator is included in the second component and preferably about 0.025 to about 3% by weight of the. activator is used.

The vehicle used to prepare the individual components of the oral composition of the present invention is substantially the same for both components and includes water and/or a suitable humectant such as glycerin, propylene glycol, polyethylene glycol, triacetin or any suitable mixture thereof. Water is preferred as a humectant in the practice of the present invention.

Ionic surfactants are used in the preparation of oral composition components of the present invention to aid in the thorough dispersion of the composition throughout the oral cavity when applied thereto as well as to improve the cosmetic acceptability and detersive and foaming properties of the composition. Among surfactants useful in the practice of the present invention are salts of the higher alkyl sulfates and alkyl phosphates having 8 to 18 carbon atoms in the alkyl group such as sodium lauryl sulfate and sodium lauryl phosphate; sodium lauryl sulfoacetate, salts of sulfonated monoglycerides of higher fatty acids, such as sodium coconut monoglyceride sulfonate or other suitable sulfonated monoglycerides of a fatty acids of 10 to 18 carbon atoms; salts of amides of higher fatty acids, e.g., 12 to 16 carbon atom acids, with lower aliphatic amino acids, such as sodium-N-methyl-N-palmitoyl tauride, sodium N-lauroyl-N,N-myristoyl- and N-palmitoyl sarcosinates; salts of the esters of such fatty acids with isothionic acid or with glycerol monosulfate, such as the sodium salt of monosulfated monoglyceride of hydrogenated coconut oil fatty acids.

The ionic surfactant is included in the peroxide free component of the present invention at a concentration of about 0.5 to about 5.0% by weight and preferably about 1.0 to about 3.0% by weight.

Nonionic surfactants are also used in the preparation of the inventive composition. Examples of this include water soluble polyoxyethylene monoester of sorbitol with a C10-18 fatty acid ester of sorbitol (and sorbitol anhydrides), consisting predominantly of the monoester, condensed with about 10-30, preferably about 20, moles of ethyleneoxide. The fatty acid (aliphatic hydrocarbon-monocarboxylic acid) may be saturated or unsaturated, e.g. lauric, palmitic, stearic, oleic acids. Tween 20, which is a polyoxyethylene (20) sorbitan monolaurate is especially preferred. The non ionic surfactant can be included in either component at a concentration of about 0.5 to about 10.0% by weight and preferably about 1.0 to about 5.0% by weight.

Thickening or gelling agents used in do formulation of the dentifrice include nonionic polyoxyethylene polyoxypropylene block copolymers. Illustrative of polyoxyethylene polyoxypropylene block copolymers useful in the practice of the present invention include block copolymers having the formula:

HO(C2CH4O)b(C3H6O6)a(C2H4I)bH,

wherein “a” is an integer such that the hydrophobic base represented by (C3H6O6) has a molecular weight of about 2750 to 4000, “b” is an integer such that the hydrophilic portion (moiety) represented by (C2CH4O) constitutes about 70-80% by weight of the copolymer. Block copolymers of this composition are available commercially under the trademark Pluronic F type. Pluronic F127, which has a molecular weight of 4000 and contains 70% of the hydrophilic polyoxyethylene moiety is preferred in the practice of the present invention. The thickening agents are preferably present in the denitifrice in an amount within the range of about 15 to about 50 percent by weight and about 25 to about 45 percent by weight is preferred for use in the compositions of the present invention.

Compounds having anti-tartar efficacy and a capability to chelate with transition metals are essential to this invention. These agents include those known in the art e.g., water soluble salts, such as dialkali or tetra-alkali metal pyrophosphate salts such as trisodium pyrophosphate, sodium hexametaphosphate, tetrasodium diphosphate and cyclic phosphates such as sodium trimetaphosphate as well as alkali metal tripolyphosphates such as sodium tripolyphosphate, potassium tripolyphosphate. Other compounds include citrates, gluconates and phosphonates such as phosphonic acid, di and tri phosphonic acid compound or its salts for example 1-hydroxyethylidene-1,1-diphosphonic acid at a concentration of about 0.05 to about 8.0%.

Fluoride providing salts having anti-caries efficacy may also be incorporated in the oral compositions of the present invention and are characterized by their ability to release fluoride ions in water. It is preferable to employ a water-soluble salt fluoride providing about 10-5,000 ppm of fluoride ion and preferably about 1000-1500 ppmn of fluoride ion. Among these materials are water-soluble alkali metal salts, for example, sodium fluoride, potassium fluoride, sodium monofluorophosphate and sodium fluorosilicate. Sodium fluoride is the preferred fluoride-providing salts.

Any suitable flavoring, sweetening or abrasive material may also be employed in the non-peroxide component. Examples of suitable flavoring constituents are flavoring oils, e.g., oils of spearmint, peppermint, wintergreen, sassafras, clove, sage, eucalyptus, marjoram, cinnamon, lemon, and orange, and methyl salicylate. Suitable sweetening agents include sucrose, lactose, maltose, sorbitol, sodium cyclamate, perillartine, and sodium saccharin. Suitably, flavor and sweetening agents may together comprise from 0.01% to 5% or more of the preparations. Suitable abrasive materials include silicas, phosphate containing calcium compounds and other materials such as aluminum oxide known in the art.

To prepare the activator containing component of the present invention sodium fluoride, sodium pyrophosphate and 1-hydroxyethylidene-1,1-diphosphonic acid are dissolved in water. Titanium dioxide is then added and the mixing continued for 10 minutes to allow for the formation of the peroxide activation complex, finally the pH is adjusted to about 10 with sodium hydroxide in order to reduce the solubility of titanium dioxide. The mixture is then transferred to a double planetary vacuum mixer and pluronic F127 is dispersed into the mixture. The components are then blended under a vacuum of 5 mm Hg. After 10 minutes the vacuum is increased to 10 mm Hg. After 20 minutes the vacuum is increased to 20 mm Hg and finally the vacuum is increased to 30 mm Hg and mixing continued until a white homogenous mixture is obtained.

To prepare the peroxide containing component of the present invention sodium fluoride, sodium pyrophosphate and l-hydroxyethylidene-1,1-diphosphonic acid are dissolved in water. Hydrogen peroxide is then added and the mixture is then transferred to a double planetary vacuum mixer and pluronic F127 is dispersed into the mixture. The components are then blended under a vacuum of 5 mm Hg. After 10 minutes the vacuum is increased to 10 mm Hg. After 20 minutes the vacuum is increased to 20 mm Hg and finally the vacuum is increased to 30 mm Hg and mixing continued until a clear gel is obtained.

In packaging the oral composition of the present invention for sale, any convenient means for effecting the separation of the peroxygen component from the activator components before use can be utilized. For example in the packaging of dentifrice components, a single container can be compartmentalized so that the peroxygen containing dentifrice component and the activator containing component are housed in separate compartments and are dispensed simultaneously for common application to a toothbrush and not admixed until applied to the teeth. Alternatively, the peroxygen containing component and the activator containing component can be housed in separate containers from which the respective phases are dispensed for admixture just prior to use.

The following examples are further illustrative of the present invention, but it is understood that the invention is not limited thereto. All amounts and proportions referred to herein and the appended claims are by weight.

### EXAMPLE 1

| | % w/w | |
|---|---|---|
| Ingredient | Component 1 | Component 2 |
| Pluronic F127 | 30 | 30 |
| Water | 54.46 | 63.46 |
| Hydrogen peroxide (30%) | 10 | — |
| Sodium pyrophosphate | 0.3 | 0.3 |
| 1-hydroxyethylidene-1,1-diphosphonic | 1.0 | 1.0 |
| acid | | |
| Sodium Fluoride | 0.24 | 0.24 |
| Sodium Lauryl sulfate | 2.00 | 2.00 |
| Tween 20 | 2.00 | 1.00 |
| Titanium Dioxide | — | 2.00 |

Component 1 was prepared by adding sodium fluoride, sodium pyrophosphate and 1-hydroxyethylidene, 1,1-diphosphonic acid to water and stirring until the materials are dissolved. Hydrogen peroxide is then added and the mixture is then transferred to a double planetary vacuum mixer and Pluronic F127 is dispersed into the mixture. The components are then blended under a vacuum of 5 mm Hg. After 10 minutes the vacuum is increased to 10 mm Hg. After 20 minutes the vacuum is increased to 20 mm Hg and finally the vacuum is increased to 30 mm Hg and mixing continued until a clear gel is obtained. Component 2 was prepared by dissolving sodium fluoride, sodium pyrophosphate and 1-hydroxyethylidene-1,1-diphosphonic acid in water. Titanium dioxide is then added and the mixing continued for 10 minutes in order to allow for the complexation of titanium with the chelating agents or to form the peroxide activation complex. The pH is adjusted to about 10 with sodium hydroxide. The mixture is then transferred to a double planetary vacuum mixer and pluronic F127 is dispersed into the mixture. If desired an abrasive material can then be added and the components are then blended under a vacuum of 5 mm Hg. After 10 minutes the vacuum is increased to 10 mm Hg. After 20 minutes the vacuum is increased to 20 mm Hg and finally the vacuum is increased to 30 mm fig and mixing continued until a white homogenous mixture is obtained.

Tooth bleaching effectiveness of the above gels was determined by using extracted human teeth. The teeth were freshly extracted, freed of all adherent tissues and stored in a sterile saline solution. The teeth were removed and the color was measured using a chroma meter (Minolta CR221). Readings were taken in the L* a* and b* parameters. The teeth were then incubated in the various gels for an additional 30 minutes. After this period the gels were washed off and color was re-measured. The change in color (Delta E) was then calculated using the CIE L*a*b* color difference equation:

Delta E=[(delta L*)2+(delta a*)2+(delta b*)2]0.5

The results are shown in EXAMPLE 2.

### EXAMPLE 2

| | Pre-treatment | | | Post-treatment | | | |
|---|---|---|---|---|---|---|---|
| Sample | L* | a* | b* | L* | a* | b* | Delta E |
| Components 1 + 2 | 54.07 | 1.11 | 4.0 | 65.71 | −1.55 | −4.06 | 14.41 |
| Component 1 | 55.49 | −1.95 | −1 | 63.48 | −2.03 | −1.31 | 8.00 |
| Component 2 | 55.79 | −1.36 | 1.09 | 55.47 | −1.47 | −0.92 | 2.04 |
| Commercial prod. | 54.00 | −1.86 | −0.47 | 58.33 | −1.91 | 0.15 | 4.37 |

L* measures tooth lightness, the greater the number the whiter the tooth. The data shown above demonstrate that the titanium activator leads to a greater improvement in tooth lightness when compared to other samples. b* measures tooth yellowness, the smaller the value the less the yellowness. The data shows that component 1 + 2 combined have reduced yellowness to a greater extent when compared to other samples. Delta E measures overall
improvement in color. The data above again shows the greatest improvement in color compared to other products. Unexpectedly, component 1 has a greater effect upon tooth whiteness when compared to the commercial product.

What is claimed is:

1. A method of whitening stained or discolored teeth in the oral cavity which comprises applying to the teeth a two component whitening composition, which will whiten stained or discolored teeth, when applied thereto, the composition being comprised of a first component containing in a matrix, a safe amount of a peroxygen compound effective to whiten teeth, and a second component in a matrix containing an insoluble transition metal oxide completed with metal chelating agents, the components being maintained separate from each other until dispensed for application to the teeth, wherein the mixing of the components results in the activation of the peroxygen compound and providing an enhanced whitening effect upon the teeth.

2. The method of claim 1 wherein the peroxygen compound is hydrogen peroxide.

3. The method of claim 1 wherein the peroxygen compound is urea peroxide.

4. The method of claim 1 wherein the peroxygen compound is calcium peroxide.

5. The method of claim 1 wherein the peroxygen compound is sodium percarbonate.

6. The method of claim 1 wherein the peroxygen compound is sodium perborate.

7. The method of claim 1 wherein the transition metal oxide is titanium dioxide.

8. The method of claim 1 wherein the transition metal oxide is zinc oxide.

9. The method of claim 1 wherein the transition metal chelating agent is one or more selected from trisodium pyrophosphate, tetrasodium diphosphate, sodium hexametaphosphate, sodium trimetaphosphate, sodium tripolyphosphate, potassium tripolyphosphate, phosphonic acid, di and tri phosphonic acid compound or its salts for example 1-hydroxyethylidene-1,1-diphosphonic acid.

10. The method of claim 1 wherein the transition metal chelating agent is one or more selected from trisodium pyrophosphate, sodium hexametaphosphate, sodium trimetaphosphate, sodium tripolyphosphate, tetrasodium diphosphate, potassium tripolyphosphate, phosphonic acid, di and tri phosphonic acid compound or its salts, for example, 1-hydroxyethylidene-1,1-diphosphonic acid.

11. A two component whitening dentifrice composition which exhibits rapid whitening of stained or discolored teeth, which composition comprises a first dentifrice component containing a peroxygen compound and a second dentifrice component containing an insoluble Transition metal compound complexed with metal chelating agents, the first and second dentifrice components being kept separate from each other until dispensed for application to teeth.

12. The composition of claim 11 wherein the peroxygen compound is hydrogen peroxide.

13. The composition of claim 11 wherein the peroxygen compound is urea peroxide.

14. The composition of claim 11 wherein the peroxygen compound is sodium percarbonate.

15. The composition of claim 11 wherein the peroxygen compound is calcium peroxide.

16. The composition of claim 11 wherein the peroxygen compound is sodium perborate.

17. The composition of claim 11 wherein the insoluble transition metal oxide is titanium dioxide

18. The composition of claim 11 wherein the insoluble transition metal oxide is zinc oxide
